(19) United States
(12) Patent Application Publication
(10) Pub. No.: US 2026/0256854 A1
(43) Pub. Date: Sep. 3, 2026

(54) COMPOSITIONS AND METHODS FOR AMELIORATING SYMPTOMS ASSOCIATED WITH INFLAMMATORY SKIN DISORDERS

(71) Applicant: CODEX LABS CORPORATION, San Jose, CA (US)

(72) Inventors: Marc CORNELL, San Jose, CA (US); Barbara A. PALDUS, San Jose, CA (US); Mark SELKER, San Jose, CA (US); Paula SIMPSON-NILSEN, Whitby (CA); Jessica Maloh, San Jose, CA (US)

(21) Appl. No.: 19/653,337

(22) Filed: Apr. 21, 2026

Related U.S. Application Data
(63) Continuation-in-part of application No. 19/576,898, filed on Mar. 24, 2026.
Application No. 19/576,898 is a continuation of application No. 18/900,196, filed on Sep. 27, 2024, now Pat. No. 12,605,417.

(51) Int. Cl. A61K 36/03; A61K 31/164; A61K 31/4172; A61K 31/728; A61K 35/745; A61K 35/747; A61K 36/185; A61K 36/28; A61K 36/30; A61K 36/53; A61K 36/61; A61K 36/80; A61K 36/899; A61P 17/00

(52) U.S. Cl. CPC A61K 36/03; A61K 31/164; A61K 31/4172; A61K 31/728; A61K 35/745; A61K 35/747; A61K 36/28; A61K 36/30; A61K 36/53; A61K 36/577; A61K 36/61; A61K 36/80; A61K 36/899; A61P 17/00

(57) ABSTRACT

The present invention is directed to compositions and methods for ameliorating symptoms associated with inflammatory skin disorders involving applying a synergistic blend of plant-based actives containing: (1) a synergistic blend of at least: (a) Calendula officinalis stem cell extract; (b) Symphytum officinale extract; (c) a biotech-derived Haberlea rhodopensis extract; and (d) Padina pavonica thallus extract; and (2) at least one skin protectant active ingredient.

### FIELD OF THE INVENTION

The present invention generally relates to compositions and methods for treating and managing inflammatory skin disorders such as dermatitis. More particularly, the present invention provides ways for effectively treating and managing inflammatory skin disorders by addressing their symptoms which include a structurally impaired stratum corneum (SC), skin inflammation, itchiness, burning, formation of lesions, and oxidative stress.

### BACKGROUND OF THE INVENTION

Skin represents the body's first line of defense against external/environmental insults that would otherwise damage sensitive underlying tissue and organs. In addition, it also functions as a relatively water-impermeable barrier that keeps water/moisture from prematurely escaping from the body. This barrier is subject to numerous extrinsic and intrinsic negative factors that affect its appearance, physical properties, and physiological functions. Extrinsic factors include ultraviolet radiation, environmental pollution, heat, harsh surfactants, lifestyle (such as diet, exercise, or sleep quality) and mechanical stress caused by, for example, shaving or garment friction and the like. Intrinsic factors include chronological aging, a person's genetic makeup, and other biological conditions such as dermatitis, psoriasis, acne vulgaris, and Hidradenitis Suppurativa (HS).

The skin is divided into two main structural layers: the epidermis and the dermis. The vital barrier function of the skin resides primarily in the top stratum of the epidermis, the stratum corneum (SC), a.k.a. the skin barrier. The epidermis also has immunologic functions and provides some protection from UV light via its pigment system. The predominant cell type of the epidermis is the keratinocyte which represents approximately 90% of the cells comprising the epidermis. They originate in the deepest layer of the epidermis (stratum basal) and migrate up to the final barrier layer, the stratum corneum.

Keratinocytes proliferate in the basal layer and start differentiating on their way to the surface. During this process, they significantly change their morphology and start to produce keratin, cytokines, growth factors, and interleukins. They play an essential role in protection as they form a tight barrier that prevents foreign substances, including pathogens, from entering the body, while minimizing the loss of moisture, heat, and other constituents. These cells have a structural role, forming tight bonds with other cells in the epidermis and maintaining them in their locations. In addition, keratinocytes function as immunomodulators following skin injuries. At each stage of differentiation, keratinocytes express not only specific keratins, but various biological markers as well such as involucrum, loricrin, transglutaminase, filaggrin, and caspase.

In addition to their structure-forming properties, keratinocytes also interact with other skin cells such as fibroblasts, melanocytes, lymphocytes, and Langerhans cells. Crosstalk between keratinocytes and fibroblasts is essential for maintaining skin homeostasis and for ensuring a balanced wound-healing process. These two cell types communicate via paracrine signaling mechanisms, the disruption of which can lead to the presence of chronic wounds. The interaction between keratinocytes and melanocytes is also critically important for skin barrier homeostasis. Melanocytes produce melanin which absorbs UV light and prevents DNA damage to the keratinocytes which, in turn, stimulate melanocyte functions such as proliferation, differentiation, and melanogenesis. Keratinocytes also possess immunomodulatory functions that interact with lymphocytes and Langerhans cells in the skin.

Keratinocytes also play a role in wound-healing via a process known as re-epithelialization, necessary for successful wound closure. When the skin is injured, keratinocytes are activated and migrate to the wound where they start proliferating to fill/close the wound. During wound healing, interactions between keratinocytes, fibroblasts, and immune cells are critically important to the healing process.

When the skin barrier is compromised (i.e., structurally impaired) or when pathogens enter the skin, an inflammatory response is triggered. Keratinocytes actively participate in this process by expressing cytokines that transmit positive or negative signals to immune cells. They also play a role in inflammatory skin conditions like dermatitis and psoriasis by recruiting and activating dendritic cells and leukocytes.

Corneocytes are essentially “dead” keratinocytes, i.e., they are devoid of nuclei and cytoplasmic organelles. They are primarily comprised of the protein keratin and are continually shed (desquamated) and renewed from lower epidermal layers, a process commonly referred to as cell turnover.

The skin barrier is often analogized to a brick wall. The corneocytes with their resistant cell envelopes and keratin microfibrils are the bricks, whereas the layers of lipids found between the cells are the mortar. This lipid mortar is the main barrier to water prematurely escaping via the stratum corneum. Both the bricks and mortar of the stratum corneum are produced by keratinocytes at the stratum granulosum where keratinocytes release the lipids (mortar) into the spaces between the cells as they are being transformed into the corneocytes (bricks). Two proteins from the keratohyalin granules, filaggrin and loricrin, play key toles in the formation of the corneocytes. Filaggrin is an acronym for filament-aggregating protein, which is derived from profilaggrin whose production is predominantly controlled by the filaggrin (FLG) gene.

The lamellar bodies that appear at the stratum granulosum contain lipids which are released into the intercellular spaces as the stratum corneum forms. The lipids are comprised of glucosyl ceramides, cholesterol esters, and long-chain fatty acids. In the intercellular space, the glucosyl ceramides are converted to ceramides. The lipids spontaneously organize into multiple layers between the stratum corneum cells.

This lipid mortar is critically important to skin barrier function, with ceramides being vital to the organization and functioning of the barrier. The lamellar bodies of the stratum corneum release other important molecules into the inter-corneocyte spaces in addition to the lipids that form the permeability barrier. The proteolytic enzymes involved in desmosome hydrolysis as well as inhibitors of those enzymes to control rates of desquamation are released by the lamellar bodies, as are antimicrobial peptides called “defensins”, which play a role in protecting the skin from infection. Production of these peptides is increased in skin with psoriasis, but not in skin suffering from atopic dermatitis (dermatitis). This is believed to account for the increased susceptibility of dermatitis sufferers to skin infection and dermatitis-prone skin may be more likely to spread nosocomial infections.

The very specialized bricks and mortar of the stratum corneum work together to produce a covering for the skin that is both flexible and protective. When functioning properly, it defends the body against dehydration, external toxins, and bacterial assault, while protecting the more fragile keratinocytes below from mechanical disruption.

FIG. 1 depicts how the skin barrier functions when healthy vs. when impaired. As can be seen in FIG. 1, a breakdown of the skin barrier results from a breakdown in the tight adhesion of the cells in the SC, and the loss of intercellular lipids (e.g.: ceramides) thereby allowing excess egress of moisture and pronounced ingress of pathogens. What is not so clear is whether the breakdown of the skin barrier is initially caused by external/environmental influences (e.g. UV rays, pollution) or by internal influences (e.g., cytokines, chemokines and reactive oxidative stress (ROS)) residing below the epidermis.

Atopic dermatitis (AD) is a common inflammatory skin disorder. While the precise cause of AD is currently unknown, its pathophysiology is influenced by a complex interplay between skin barrier dysfunction, inflammation, genetics, and environmental factors. This pathophysiology can be reduced to a basic description which generally consists of three fundamental steps, though not necessarily in this precise order given the two competing theories for the genesis of these diseases. In the “outside-in” hypothesis, epidermal barrier dysfunction triggers immune activation; in contrast, based on the “inside-out” hypothesis, dermatitis is primarily cytokine driven with secondary skin barrier dysfunction.

The modern approach to defining dermatitis pathogenesis appears to now be centered on integrating these two mechanisms and is oriented toward characterizing their interplay in AD as it is not clear which individual model is implicated or whether a combination of the two models explains the disease process. (Facheris, P., Jeffery, J., Del Duca, E. et al. The translational revolution in atopic dermatitis: the paradigm shift from pathogenesis to treatment. Cell Mol Immunol 20, 448-474 (2023). doi.org/10.1038/s41423-023-00992-4.)

Myriad central genetic mutations have been demonstrated in the pathogenesis of AD: mutations in structural epidermal barrier proteins, mutations in functional proteins that maintain the epidermal barrier, and mutations in factors that regulate the immune system. Skin barrier alterations, with dysfunction of barrier-related proteins such as filaggrin (FLG), loricrin (LOR), and involucrin (IVL), are the first steps that explain the subsequent sensitization against allergens and the so-called “atopic march” in the “extrinsic” form of AD, where the extrinsic form of AD has a signature increase in total serum IgE levels. Alteration of the skin barrier is also related to alteration of the inter-corneocyte lipid composition (e.g. ceramides), with further trans-epidermal water loss (TEWL). This, in turn, contributes to the skin dryness typical of AD patients and increased penetration of allergens and pathogens creating a feedback loop.

Skin barrier dysfunction in AD is characterized by a lower expression of terminal differentiation markers, such as filaggrin (FLG) and loricrin (LOR), and by a higher permeability defect caused by skin lipid film impairment and higher trans-epidermal water loss. This makes AD skin more prone to the penetration of external agents (antigens, allergens, pollution, etc.) that are harmful to keratinocytes. Damaged keratinocytes produce epidermal alarmins such as IL-33, IL-25, and TSLP, which activate the dendritic cells (DCs) and type 2 innate lymphoid cells (ILC2s) that produce IL-5 and IL-13, which activate eosinophils and Th2 cells. Local Th2 polarization, in return, further diminishes barrier functions and sustains itching, causing skin barrier impairment and facilitating dysbiosis. AD skin shows a higher proliferation of the members of the genus Staphylococcus, especially S. aureus, which can further damage keratinocytes and sustain local inflammation.” (Facheris, P., Jeffery, J., Del Duca, E. et al. The translational revolution in atopic dermatitis: the paradigm shift from pathogenesis to treatment. Cell Mol Immunol 20, 448-474 (2023). doi.org/10.1038/s41423-023-00992-4.)

Conversely, while sharing a similar Th2 signature, “intrinsic” AD is characterized by increased Th1 expression in the blood compared to extrinsic AD and lower CCL17 levels. Intrinsic AD also shows greater cellular infiltrates with T cells, Langerhans cells, and myeloid DCs. Th17- and Th22-driven expression of the antimicrobials S100A9 and S100A12 was found to be higher in intrinsic AD, with Th17 expression positively correlating with disease severity. (Facheris, P., Jeffery, J., Del Duca, E. et al. The translational revolution in atopic dermatitis: the paradigm shift from pathogenesis to treatment. Cell Mol Immunol 20, 448-474 (2023). doi.org/10.1038/s41423-023-00992-4.)

The microenvironment of a compromised skin barrier contributes, especially in the acute phase, to the activation of the immune system with a predominantly, but not exclusively Th2-mediated reaction and the release of proinflammatory cytokines and chemokines. On the other hand, this process may occur in the opposite order with proinflammatory processes leading to a disturbed skin barrier. Either way, these cytokines and chemokines include alarmins such as tumor necrosis factor (TNF) and interleukins (IL-4, IL-9, IL-22). Epithelium-derived cytokines, such as thymic stromal lymphopoietin (TSLP), IL-25 and IL-33 also act as alarmins after appropriate stimulation, including oxidative stress (OS), and activate a Th2-mediated response in AD, contributing to the inflammatory state of the skin.

The persistence of Th2 inflammation and skin barrier disruption contributes to inflammation and to the overproduction of reactive oxygen species (ROS), such as superoxide and hydrogen peroxide. In addition to this mechanism, the increase in ROS may also depend on other exogenous factors such as solar radiation, pollution, psychological stress, and infections. For example, Staphylococcus aureus, a pathogen that frequently causes skin infections and flares up in patients with AD because of a disruption in the skin barrier such as in the case of filaggrin deficiency, can sustain skin inflammation through ROS released by monocytes activated by the pathogen itself. Over time, the accumulation of ROS can eventually cause oxidative stress due to an imbalance between the generation of excess ROS and the body's ability to produce ROS-neutralizing antioxidants.

Inflammatory skin disorders characterized by itch as the primary symptom encompass a wide range of conditions, each with its unique underlying causes and manifestations. These disorders, collectively referred to as inflammatory itch disorders, often present with intense itching sensations, leading to significant discomfort and distress for affected individuals. Conditions such as atopic dermatitis, psoriasis, and allergic contact dermatitis are prime examples of inflammatory skin disorders where itch plays a central role. The itching sensation associated with these conditions can be relentless, interfering with daily activities, disrupting sleep patterns, and negatively impacting quality of life. Despite varying etiologies, these disorders share common inflammatory pathways that contribute to the perception of itch, including activation of immune cells, release of proinflammatory mediators, and alteration in nerve signaling within the skin.

One key player in the pathogenesis of inflammatory itch disorders, particularly in children, is the gene interleukin-13 (IL-13). IL-13 is a cytokine, i.e., a small protein involved in cell signaling, that plays a crucial role in regulating immune responses and inflammation. In the context of inflammatory skin conditions, IL-13 has been implicated in promoting the inflammatory cascade that triggers the itch sensation. Research has shown that elevated levels of IL-13 are present in the skin of individuals with conditions like atopic dermatitis, where itch is a predominant symptom.

Genetic studies have identified variation in the IL-13 gene that may predispose individuals, especially children, to develop inflammatory skin disorders characterized by severe itching. The overproduction of IL-13 triggers a cascade of events within the skin, including the recruitment of immune cells and the release of itch-inducing molecules, ultimately leading to heightened itch perception and exacerbation of the underlying skin inflammation.

In individuals suffering from inflammatory disorders such as dermatitis and psoriasis, not only is their skin barrier compromised leading to increased trans epidermal water loss (TEWL) and enhanced susceptibility to skin irritants and allergens, but so too is their skin's microbiome. In the case of dermatitis, there is often a reduced diversity of microbial species and an overgrowth of certain harmful bacteria such as Staphylococcus aureus. More particularly, during AD flare-ups, the loss of microbiome diversity towards an overgrowth of S. aureus correlates with disease severity. S. aureus strains isolated from AD lesions have been shown to produce a variety of toxins and enzymes with aggressive cell-damaging and inflammation-inducing properties. S. aureus directly damages keratinocytes by adhering to cells and forming transmembrane pores through the secretion of staphylococcal toxin ultimately leading to the breakdown of cellular ATP metabolism. S. aureus superantigens elicit the production of IgE antibodies, levels of which correlate with disease severity. This imbalance, known as dysbiosis, exacerbates inflammation and thereby contributes to the severity of dermatitis symptoms.

When it comes to treatment options, topical corticosteroids are overwhelmingly the most frequently prescribed class of drugs for atopic dermatitis. However, long-term application of topical corticosteroids is not recommended because of the risk of skin atrophy, dyspigmentation, acneiform eruptions, and risks associated with systemic absorption such as Cushing's disease. Topical calcineurin inhibitors are generally effective and safe as short-term treatments, but concerns of skin malignancies and increased risk of lymphomas have prompted regulatory authorities to require a warning regarding their long-term safety.

First generation antihistamines are widely prescribed for acute symptomatic treatment of pruritus, although their effectiveness is limited and largely attributed to their sedating effect. Oral immunosuppressants and glucocorticoids are effective but are sometimes associated with severe toxicity and side effects, thus limiting their use to short courses and/or intermittent therapy. No systemic agents are approved in the treatment of atopic dermatitis in children. All systemic agents are used off label (cyclosporine, methotrexate, etc.) and have a broad immunosuppressive effect which predisposes the patients to serious infections and increased risk of malignancies if used for prolonged periods. Other reported significant side effects with these agents include gastritis, stunted growth, diabetes, weight gain, hypertension, osteoporosis, and adrenal suppression (corticosteroids), elevated liver enzymes and leukopenia. Moreover, a high proportion of patients in which the condition is initially controlled by systemic agents suffer from relapse once therapy is discontinued. As a result, there exists a significant unmet need for alternative compositions and methods for managing inflammatory skin conditions such as dermatitis and psoriasis.

Based on the foregoing, it is an object of the present invention to provide compositions and methods capable of addressing the symptoms associated with chronic inflammatory skin disorders which include at least one of the following: (i) a compromised/structurally degraded skin barrier, (ii) inflammation caused by the activation of the body's immune system and its release of proinflammatory cytokines, (iii) itching sensation, (iv) burning sensation, (v) formation of lesions on the skin surface, and (vi) oxidative stress, in order to effectively treat and manage such disorders.

It is also an object of the present invention to provide methods of treating and managing inflammatory skin disorders by effectively addressing the above-referenced symptoms.

The pathophysiology of AD is multifactorial, encompassing genetic predisposition, immune dysregulation, and structural deficiencies in the epidermal barrier. AD affects millions of individuals worldwide across all age groups, and its prevalence continues to rise.

A contributor to AD pathogenesis is the deficiency of filaggrin, a structural protein encoded by the FLG gene. Filaggrin performs two essential functions in the stratum corneum: it organizes keratin filaments within differentiating keratinocytes to form the compact, flattened corneocytes that constitute the “bricks” of the epidermal barrier, and it is subsequently proteolytically degraded into hygroscopic amino acids and related compounds that collectively form the natural moisturizing factor (NMF). NMF plays a role in maintaining stratum corneum hydration, barrier integrity, controlled desquamation, and an appropriately acidic surface pH.

Loss-of-function mutations in the FLG gene represent the strongest known genetic risk factor for AD, resulting in reduced filaggrin expression, diminished NMF, elevated transepidermal water loss (TEWL), and increased susceptibility to allergens, irritants, and microbial colonization. However, it is also well-known that use of topical steroid creams for inflammatory skin conditions reduce filaggrin production after about 4 weeks of continuous use. Systemic inflammation reduces filaggrin (FLG) production primarily by releasing inflammatory cytokines into the bloodstream, which then act on keratinocytes within the skin to suppress the expression of the FLG gene and inhibit the processing of its precursor, profilaggrin. Filaggrin processing is also decreased by the downregulation of caspase-14, which processes filaggrin to profilaggrin. This process leads to acquired filaggrin deficiency, which weakens the skin barrier and enables increased allergen penetration.

Beyond direct FLG mutations, filaggrin expression is regulated at the transcriptional level by OVOL1, a zinc-finger transcription factor that governs epidermal differentiation. The IL-13-OVOL1-FLG axis plays a key role in AD pathogenesis: elevated IL-13 levels, characteristic of Th2-skewed atopic inflammation, suppress OVOL1 expression, which in turn reduces filaggrin production and weakens the skin barrier. Mechanistically, activation of the aryl hydrocarbon receptor (AHR) drives translocation of OVOL1 from the cytoplasm into the nucleus, where it promotes FLG gene expression. Compounds that activate the AHR-OVOL1-FLG axis have accordingly been shown to restore filaggrin expression and repair barrier impairment. OVOL1 and filaggrin deficiencies are not limited to AD; reduced levels of both proteins are also observed in psoriatic skin relative to healthy skin, underscoring their broader significance in inflammatory barrier disorders. Upregulation of OVOL1 therefore represents a compelling therapeutic target for improving barrier function and filaggrin levels in atopic and related skin conditions.

L-histidine is an essential amino acid that serves as a key structural building block of the filaggrin protein. Supplemental L-histidine provides the skin with additional raw material to support filaggrin biosynthesis in keratinocytes that retain partial FLG gene function. In vitro studies have confirmed that L-histidine enhances filaggrin processing in cultured human keratinocytes and strengthens barrier function in human skin equivalents. Clinical investigations employing L-histidine in subjects with AD (for example, daily oral L-histidine doses of 4 g in adults and 0.8 g in children with mild to moderate AD) have demonstrated statistically significant reductions in AD severity after 4 to 12 weeks of use.

While dietary supplementation with L-histidine has been shown to help enhance filaggrin biosynthesis in human skin, upregulation of the FLG gene via this treatment strategy takes time to be realized, leaving the skin barrier compromised and eczema-related symptoms active in the interim. The ability to accelerate FLG upregulation would enhance skin barrier repair while bringing relief from eczema-related symptoms in a speedier fashion.

It is therefore an object of the present disclosure to provide a method of accelerating upregulation of the FLG gene in a subject having skin suffering from a chronic inflammatory skin disorder, eczema-prone skin, and/or skin suffering from a FLG gene loss-of-function mutation. It is a further object of the present disclosure to provide a method of stimulating filaggrin production in the skin of the subject.

It is therefore another object of the present disclosure to provide a method of upregulating OVOL1 in order to further stimulate FLG production in a subject having skin suffering from a chronic inflammatory skin disorder, atopic dermatitis, and/or FLG gene loss-of-function mutation.

### SUMMARY OF THE INVENTION

According to certain aspects of the present invention, compositions and methods are provided for treating and managing symptoms associated with inflammatory skin disorders such as dermatitis and psoriasis.

According to one embodiment, the present disclosure provides a topical skincare composition containing: (1) a synergistic blend of at least: (a) Calendula officinalis plant or stem cell extract; (b) Symphytum officinale extract; (c) a Haberlea rhodopensis extract, e.g., a biotech-derived Haberlea rhodopensis extract; (d) Padina pavonica thallus extract; and (2) at least one skin protectant active ingredient, wherein the composition is capable of effectively treating and managing symptoms associated with inflammatory skin disorders such as dermatitis and psoriasis.

In some embodiments, (a) is present in an amount of from about 0.1 to about 5% by weight, (b) is present in an amount of from about 0.1 to about 5% by weight, (c) is present in an amount of from about 0.1 to about 5% by weight, (d) is present in an amount of from about 0.1 to about 5% by weight; and (2) is present in an amount of from about 0.01 to about 5% by weight, all weights based on total weight of the composition.

In some embodiments, (a) is present in an amount of from about 0.5 to about 4% by weight, (b) is present in an amount of from about 0.1 to about 4% by weight, (c) is present in an amount of from about 0.5 to about 4% by weight, (d) is present in an amount of from about 0.5 to about 4% by weight; and (2) is present in an amount of from about 0.02 to about 3% by weight, all weights based on total weight of the composition.

In some embodiments, (a) is present in an amount of from about 1 to about 3% by weight, (b) is present in an amount of from about 0.4 to about 3% by weight, (c) is present in an amount of from about 0.75 to about 2% by weight, (d) is present in an amount of from about 1 to about 3% by weight; and (2) is present in an amount of from about 0.5 to about 2% by weight, all weights based on total weight of the composition.

In some embodiments, the composition further includes at least one humectant. In some embodiments, the at least one humectant is present in an amount of from about 0.1 to about 10% by weight, or from about 4 to about 8% by weight, based on total weight of the composition. In some embodiments, the at least one humectant is hyaluronic acid.

In some embodiments, the composition further includes at least one ceramide. In some embodiments, the ceramide is present in an amount of from about 0.01 to about 0.5% by weight, or from about 0.02 to about 0.3% by weight, based on total weight of the composition.

In some embodiments, the present disclosure provides a method of treating and managing symptoms associated with inflammatory skin disorders, comprising applying the composition described herein onto skin of a subject in need thereof. In some embodiments, the symptoms include at least one of a compromised skin barrier, inflammation, itchiness sensation, burning sensation, formation of lesions, and oxidative stress.

In some embodiments, the method further includes oral administration of a dietary supplement comprising at least: (a) Buddleja globosa leaf extract; (b) Aristotelia chilensis leaf extract; (c) Ugni molinae leaf extract; and (d) L-histidine.

In some embodiments, the method may also include cleansing skin with a microbiome-friendly cleansing product prior to applying the topical composition onto the skin.

In some embodiments, the method may further include applying a bacteriophage topical product that is designed to kill different S. aureus species. The bacteriophage will preferably be applied onto the skin prior to application of the composition of the present disclosure.

According to yet another embodiment, the method further includes orally administering a synbiotic supplement comprising a therapeutically effective amount of: (a) a probiotic blend of bacterial components comprising at least: (i) Lactobacillus helveticus, (ii) Lactobacillus plantarum, (iii) Bifidobacterium longum, and (iii) Streptococcus thermophilus; and (b) at least one plant-based, antioxidant-rich source of fiber.

In yet another embodiment, the method of the present disclosure further includes topical application of a probiotic serum comprising live Lactobacillus plantarum lactobacilli.

In yet another embodiment, the present disclosure is directed to a method of stimulating filaggrin production in skin of a subject suffering from a chronic inflammatory skin disorder and/or suffering from a loss of function mutation in gene FLG, the method comprising: (i) applying a topical composition onto the skin of the subject, the composition comprising: (1) a synergistic blend of at least: (a) Calendula officinalis extract; (b) Symphytum officinale extract; (c) Haberlea rhodopensis extract; and (d) Padina pavonica thallus extract; and (2) a skin protectant active ingredient, and (ii) orally administering a dietary supplement to the subject, the dietary supplement comprising L-histidine. In some embodiments, the composition is capable of increasing ceramide production and reducing permeability of the skin barrier.

In yet another embodiment, the present disclosure is directed to a method of stimulating FLG gene synthesis in skin of a subject suffering from a chronic inflammatory disorder and/or a loss-of-function mutation in gene FLG, the method comprising: (i) topically applying onto the skin of the subject a composition containing: (1) a synergistic blend of at least: (a) Calendula officinalis plant or stem cell extract; (b) Symphytum officinale extract; (c) a Haberlea rhodopensis extract, e.g., a biotech-derived Haberlea rhodopensis extract; (d) Padina pavonica thallus extract; and (2) at least one skin protectant active ingredient; and (ii) administering a dietary supplement containing L-histidine to the subject. In some embodiments, the composition is capable of increasing ceramide production and reducing permeability of the skin barrier. In some embodiments, steps (i) and (ii) are performed on a daily basis.

In another embodiment, the present disclosure is directed to a method of stimulating FLG and/or OVOL1 gene synthesis in skin of a subject suffering from a chronic inflammatory disorder and/or a loss-of-function mutation in gene FLG, the method comprising: (i) topically applying onto the skin of the subject a composition containing: (1) a synergistic blend of at least: (a) Calendula officinalis plant or stem cell extract; (b) Symphytum officinale extract; (c) a Haberlea rhodopensis extract, e.g., a biotech-derived Haberlea rhodopensis extract; (d) Padina pavonica thallus extract; and (2) at least one skin protectant active ingredient; and (ii) administering a dietary supplement containing L-histidine and an antioxidant to the subject. In some embodiments, the composition is capable of increasing ceramide production and reducing permeability of the skin barrier. In some embodiments, steps (i) and (ii) are performed on a daily basis.

In further embodiments, the present disclosure is directed to a kit comprising (i) a composition comprising (1) a synergistic blend of at least: (a) Calendula officinalis extract; (b) Symphytum officinale extract; (c) Haberlea rhodopensis extract; and (d) Padina pavonica thallus extract; and (2) a skin protectant active ingredient; and (ii) a dietary supplement comprising L-histidine.

Other embodiments of the present disclosure will become apparent from a review of the ensuing detailed disclosure.

### BRIEF DESCRIPTION OF THE DRAWINGS

To describe the manner in which the above-recited and other advantages and features of the disclosure can be obtained, a more particular description of the disclosure briefly described above will be rendered by reference to specific embodiments thereof, which are illustrated in the appended drawings.

FIG. 1 depicts the functioning of a healthy skin barrier versus a structurally damaged one. FIG. 1 is from www.elysiumhealth.com/blogs/aging101/what-is-the-skin-barrier.

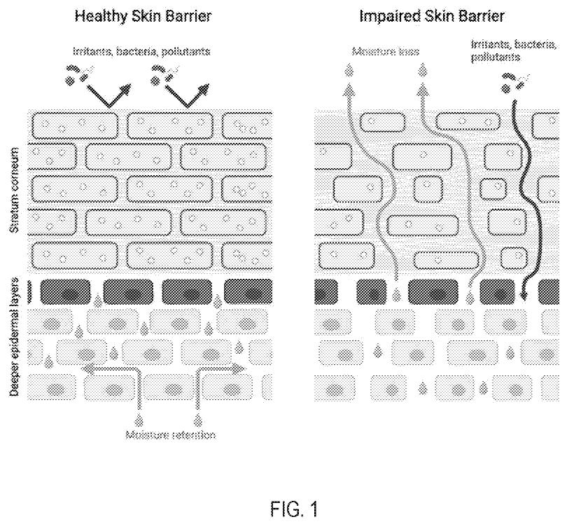

### DETAILED DESCRIPTION

For purposes of the present disclosure, the use of the term “inflammatory skin disorders” is intended to encompass those disorders whose symptoms include at least one of: a structurally impaired skin barrier; inflammation; itchiness sensation; burning sensation; formation of lesions, and oxidative stress. Examples include dermatitis, psoriasis, and hidradenitis suppurativa.

Compositions of the present disclosure can comprise, consist essentially of, or consist of, the components of the present disclosure as well as other ingredients described herein. The term “comprising” as used herein is meant to include various optional, compatible components that can be used in the preservative systems and cosmetic compositions of the present disclosure without limiting the inclusion, use of, or cooperation with other ingredients, excipients, uses, or otherwise. The term “consisting essentially of” as used herein means that the composition or component may include additional ingredients, but only if the additional ingredients do not materially alter the basic and novel characteristics of the compositions or methods.

As used herein, the words “preferred,” “preferably,” and variants thereof refer to embodiments of the disclosure that afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the disclosure.

Numerical ranges as used herein are intended to include every number and subset of numbers contained within that range, whether specifically disclosed or not. Further, these numerical ranges should be construed as providing support for a claim directed to any number or subset of numbers within that range.

All percentages, parts, proportions, and ratios as used herein are by weight of the total composition, unless otherwise specified. All such weights as they pertain to listed ingredients are based on the active level.

All references to singular characteristics or limitations of the present disclosure shall include the corresponding plural characteristic or limitation, and vice versa, unless otherwise specified or clearly implied to the contrary by the context in which the reference is made.

All publications, articles, papers, patents, patent publications, and other references cited herein are hereby incorporated in their entireties for all purposes to the extent consistent with the disclosure herein.

The term “oxidative stress” as described herein refers to the disturbance in balance between reactive oxygen species (ROS) and antioxidants present in the skin caused by extrinsic and/or intrinsic factors. Extrinsic factors include, for example, exposure to UV radiation, high energy visible light, pollution, and products containing harsh chemicals. Intrinsic factors include, for example, chronological aging, a person's genetic makeup, and other biological changes that occur from within the skin such as atopic dermatitis, psoriasis, acne vulgaris, and hidradenitis suppurativa.

The term “consistently applying” as described herein refers to an individual suffering from inflammatory skin disorders incorporating the composition of the present disclosure into their skincare regimen.

The term “microbiome-friendly” as described herein refers to a product which is capable of preserving microbial population, diversity and balance on the skin.

The term “bacteriophage” as used herein refers to those viruses capable of killing Staphylococcus aureus (S. aureus) bacterium.

As used herein, “treatment” or “treating” means the alleviation, prophylaxis or reversal of a condition, a disease, or a disorder, or at least a discernible symptom thereof. In one embodiment, “treatment” or “treating” refers to a mitigation, prophylaxis, or reversal of at least one measurable physical parameter related to the condition, disease, or disorder being treated, not necessarily discernible in or by the individual being treated. In yet another embodiment, “treatment” or “treating” refers to inhibiting or slowing the progression of a condition, a disease, or a disorder, either physically, e.g., stabilization of a physiologically discernible symptom, e.g., stabilization of a physical parameter, or both. In yet another embodiment, “treatment” or “treating” refers to delaying the onset of a condition, disease, or disorder.

As used herein, the term “probiotics” is defined as substances which stimulate the growth of microorganisms, especially those with beneficial properties that give health benefits to the host when orally administered in an appropriate amount. Their established safety and beneficial effects on human health have led to the emergence of probiotics as substitutes or complements to medicines. The advantage of probiotics is that they have very few side effects.

The term “prebiotics” typically refers to foods having a high fiber content which can act as a food source for human microflora.

As used herein, the term “synbiotics” refers to dietary supplements comprised of both prebiotic and probiotic ingredients, the combination of which has immunomodulating and gastrointestinal flora-restoring activity.

It has surprisingly and unexpectedly been discovered by the inventors that a combination of: (a) Calendula officinalis extract; (b) Symphytum officinale extract; (c) Haberlea rhodopensis extract, (d) Padina pavonica thallus extract, and (e) at least one skin protectant active ingredient, when applied onto skin, has a synergistic influence on the skin barrier and, in particular, certain genes that are known to help manage oxidative stress, restore skin barrier structure and integrity, reduce inflammation, restore skin barrier homeostasis, and support wound healing and tissue remodeling. The synergistic influence on these genes facilitates the effective treatment and management of certain symptoms associated with inflammatory skin disorders such as dermatitis, psoriasis, seborrheic dermatitis, and hidradenitis suppurativa. In some embodiments, the composition is capable of increasing ceramide production and reducing permeability of the skin barrier.

Calendula officinalis extract is derived from the cells of the marigold plant. Common bioactive compounds found therein include, for example, flavonoids, phenolic acids, carotenoids, triterpenic alcohols, polycarbohydrates, proteins, amino acids, saturated hydrocarbons, vitamin C, and mineral substances. One example of the type of Calendula officinalis extract used by the present disclosure is commercially available from Innova BM, a company headquartered in Bulgaria, which sells the extract under the tradename Innova Stem Cell Calendula W®.

In some embodiments, the Calendula officinalis extract is a stem cell extract. The Calendula officinalis stem cell extract of the present disclosure may be employed in an amount of from about 0.1 to about 5% by weight, including from about 0.5 to about 4% by weight, or from about 1 to about 3% by weight, or from about 2 to about 3% by weight, all weights based on the total weight of the composition. The Calendula officinalis extract is employed in the form of an oil infusion derived from either a plant macerate or plant stem cells grown in a bioreactor. The biotech method employed by Innova BM uses an in-vitro cell culture of Calendula officinalis cells to create a sustainable, eco-friendly cell extract containing a higher concentration of beneficial phyto-chemicals.

Symphytum officinale extract comprises the primary bioactive compounds found in Symphytum officinale, including allantoin, rosmarinic acid and ellagic acid.

The Symphytum officinale extract of the present disclosure may be employed in an amount of from about 0.1 to about 5% by weight, including from about 0.1 to about 4% by weight, or from about 0.4 to about 3% by weight, or from about 0.4 to about 2% by weight, all weights based on the total weight of the composition. In some embodiments, the Symphytum officinale extract is employed in the form of an oil infusion derived from either a plant macerate or plant stem cells grown in a bioreactor. The biotech method uses an in-vitro cell culture of Symphytum officinale cells to create a sustainable, eco-friendly cell extract containing a higher concentration of beneficial phyto-chemicals. One example of the type of Symphytum officinale extract used in the present disclosure is commercially available from O&3, headquartered in England, in the form of an oil infusion.

Haberlea rhodopensis is a tertiary relict plant endemic to the Balkan region, especially the Rhodope mountains in Bulgaria. In some embodiments, the Haberlea rhodopensis extract of the present disclosure is employed in the form of an oil infusion generated from either a plant macerate or plant stem cells grown in a bioreactor. A Haberlea rhodopensis extract produced from plant macerate or plant stem cells grown in a bioreactor is referred to herein as a “biotech-derived Haberlea rhodopensis extract.”

The Haberlea rhodopensis extract of the present disclosure may be employed in an amount of from about 0.1 to about 5% by weight, including from about 0.5 to about 4% by weight, or from about 0.75 to about 2% by weight, all weights based on the total weight of the composition.

One example of the type of Haberlea rhodopensis extract used in the present disclosure is commercially available from various suppliers including, for example, Innova BM, a company headquartered in Bulgaria, under the tradename InnovaBio Tech Haberlea®. The biotech method employed by Innova BM uses an in-vitro cell culture of Haberlea rhodopensis cells cultivated from the plant to create a sustainable, eco-friendly cell extract containing a higher concentration of beneficial phyto-chemicals.

Padina pavonica thallus extract is derived from a brown alga found in the Mediterranean Sea. The algal extract is typically obtained from an aqueous based extraction under pressure. This ingredient is commercially available from Biosil Technologies, Inc., a company headquartered in Allendale, NJ, under the tradename Ocea Health®. The Padina pavonica thallus extract of the present disclosure may be employed in an amount of from about 0.1 to about 5% by weight, or from about 0.5 to about 4% by weight, or from about 1 to about 3% by weight, all weights based on the total weight the composition.

Examples of suitable skin protectant active ingredients which may be employed include those found in the FDA's OTC Monograph M016, the entire contents of which is hereby incorporated by reference. Exemplary skin protectant active ingredients include, but are not limited to, colloidal oatmeal and allantoin. In some embodiments, the composition of the present disclosure includes a skin protective active ingredient selected from colloidal oatmeal, allantoin, and mixtures thereof.

The skin protectant active ingredient of the present disclosure may be employed in an amount of from about 0.01 to about 5% by weight, or from about 0.02 to about 3% by weight, or from about 0.5 to about 2% by weight, all weights based on the total weight the composition.

According to another embodiment of the present disclosure, the composition further includes at least one humectant. In some embodiments, the humectant provides enhanced hydration and moisturization to de-hydrated skin effected by an inflammatory skin disorder. The humectant may be employed in an amount of from about 0.05 to about 10% by weight, from about 0.1 to about 10% by weight, or from about 4 to about 8% by weight, all weights based on the total weight of the composition. Examples of suitable humectants include, but are not limited to, hyaluronic acid and its derivatives such as sodium hyaluronate, hydrolyzed sodium hyaluronate, and hydrolyzed hyaluronic acid, lecithin, aloe vera, panthenol, glycerin, and seaweed. A particularly preferred humectant for use in embodiments of the present disclosure is hyaluronic acid. In some embodiments, the humectant is hydrolyzed hyaluronic acid, sodium hyaluronate, hydrolyzed sodium hyaluronate, or a combination thereof.

According to another embodiment of the present disclosure, the composition further includes at least one ceramide. In some embodiments, the ceramide structurally enhances the stratum corneum. Ceramides are a group of natural waxy, fatty substances in the skin, composed of sphingosine and lipids (fatty acids) bonded together. Ceramides make up about 50% of all skin lipids and are manufactured in the lower, living cells of the epidermis. As the cells mature and move to the surface, ceramides are released to the topmost layer, the stratum corneum. In the stratum corneum layer, ceramides combine with cholesterol (another important lipid found in the skin) and fatty acids to form an ordered, tightly packed, layered, sheet-like arrangement between the dead cells. Ceramides and cholesterol protect against moisture loss to keep skin youthful and supple and support the structure of the stratum corneum. Exemplary ceramides include ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate.

The ceramide may be employed in an amount of from about 0.01 to about 0.5% by weight, from about 0.05 to about 0.5% by weight, from about 0.1 to about 0.5% by weight, or from about 0.2% to about 0.3% by weight, all weights based on the total weight of the composition, it being understood that the ceramide component of the composition may comprise a mixture of two or more different ceramides. In some embodiments, the at least one ceramide is selected from ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, ceramide NS dilaurate, or a combination thereof.

In yet another embodiment of the present disclosure, the compositions are free of a skin-sensitizing amount of an essential oil. In some embodiments, the compositions have a pH ranging from about 4.5 to about 5.5, and preferably from about 4.8 to about 5.3. In some embodiments, a composition “free of” a particular component means that the composition includes less than about 1% by weight, or less than about 0.8% by weight, or less than about 0.5% by weight, or less than about 0.2% by weight, or less than about 0.1% by weight, or less than about 0.05% by weight, or less than 0.01% by weight of the particular component.

In another embodiment, the present disclosure provides a composition containing: (1) a therapeutic blend of plant-based actives comprising at least: (a) Calendula officinalis extract; (b) Symphytum officinale extract; (c) Haberlea rhodopensis extract; and (d) Padina pavonica thallus extract; (2) at least one skin protectant active ingredient; (3) at least one humectant; (4) at least one ceramide; (5) an emulsifier; and (6) a dermatologically acceptable carrier, wherein the composition is capable of effectively treating and managing symptoms associated with dermatitis. In some embodiments, the composition is capable of increasing ceramide production and reducing permeability of the skin barrier.

Any ingredient capable of emulsifying the composition may be employed as an emulsifier without departing from the spirit of the invention, so long as it is natural and dermatologically acceptable. Examples thereof include, but are not limited to, glyceryl stearate, cetyl alcohol, sodium stearoyl lactylate, sorbitan olivate, cetearyl olivate, cetearyl alcohol, cetearyl glucoside, sodium cetearyl sulfate, and the like. It is also particularly preferred that the emulsifier be free of palm oil.

The compositions of the present disclosure may be in a wide variety of product forms that include, but are not limited to, solutions, suspensions, lotions, creams, gels, sprays, foams, ointments, and serums.

According to embodiments of the present disclosure, the compositions can also additionally comprise suitable optional ingredients as desired. For example, the composition can optionally include other active or inactive ingredients, provided they do not unacceptably alter the benefits of the composition and do not promote skin sensitization. The precise amount of optional ingredients chosen will be determined by those skilled in the art.

Examples of optional ingredients that may be employed include, but are not limited to, emollients, flavonoids, minerals, chelating agents, pH regulators/buffers, rheology modifiers, phytosterols, vitamin B12 compound, vitamin D3 compound, anti-inflammatory agents such as licorice extracts, bisabolol, manjistha extracted from plants in the genus Rubia, guggul extracted from plants in the genus Commiphohra, Quillaja saponaria extract, kola extract, chamomile, red clover extract, sea whip extract, hibiscus extract, maqui extract, meadowsweet extract, rosemary extract, lucuma extract, sea kale extract, Iceland Moss extract, Saskatoon Berry extract, Siberian Ginseng extract, spruce needles extract, birch bark extract, blueberry extract, cranberry extract, yarrow extract, marigold extract, and couch grass extract.

A preferred optional ingredient that may be employed is an emollient such as, for example, squalane and/or hemi-squalane.

Additional ingredients that may be employed in order to further potentiate the invention's efficacy may include, for example, Astrocaryum murumuru seed butter, Theobroma grandiflorum seed butter, Theobroma grandiflorum seed butter, Spondias mombin pulp extract, Mangifera indica pulp extract, Musa sapientum pulp extract, Mauritia flexuosa fruit oil, Physalis angulata extract, Xylityl sesquicaprylate, Vaccinium myrtillus seed oil, Cucubita pepo seed extract, linoleic acid, linolenic acid, Centella asiatica leaf extract, Tamarindus indica see polysaccharide, Zanthoxylum bungeanum fruit extract, fucoidan extract, allantoin, Lactococcus ferment lysate, Bellis perennis flower extract, Coffea arabica seed cake extract, Coffea arabica seed oil, cotton seed oil, sunflower seed oil, almond oil, linseed oil, Pichia ferment lysate filtrate, and whey protein.

The dermatologically acceptable carrier can encompass a wide variety of forms. In some cases, the solubility or dispersibility of the components in the composition may dictate the form and character of the carrier. Non-limiting examples include simple solutions (e.g., aqueous or anhydrous), dispersions, emulsions and solid forms. In certain embodiments, the dermatologically acceptable carrier is in the form of an emulsion. An emulsion can be generally classified as having a continuous aqueous phase (e.g., oil-in-water and water-in-oil-in-water) or a continuous oil phase (e.g., water-in-oil or oil-in-water). While the oil phase may comprise any vegetable oil, so long as it does not cause skin sensitization, a particularly preferred oil component is sunflower seed oil.

The inventors have surprisingly discovered that the use of sunflower oil enables bioactive compounds present in the composition to effectively penetrate into the skin, without the need of having to use skin-sensitizing essential oils, while still facilitating the desired degree of efficacy. The high concentration of linoleic acid (at least 60%) helps maintain the skin barrier and serve as an emollient to lock in moisture and reduce trans-epidermal water loss (TEWL). Sunflower oil is also rich in vitamin E, an antioxidant that helps protect the skin barrier from external stressors like sun exposure and air pollution, vitamins C and D as well as beta-carotene (pre-cursor to vitamin A) that can combine with fatty acids to promote new skin cell growth and prevent of bacterial infection and omega-6 fatty acids that help reduce redness and smooth roughness. In addition, sunflower oil is safe for people with tree nut allergies. Other vegetable oils that may also be used include, but are not limited to, almond oil, olive oil, jojoba oil, babassu oil, castor oil, coconut oil, corn oil, cotton seed oil, linseed oil, mustard oil, safflower oil, sesame oil, soybean oil, sunflower oil, wheat germ oil, argan oil and marula oil.

According to another embodiment, there is provided a method of treating and managing symptoms associated with dermatitis by applying one of the above-disclosed compositions onto the skin of a subject in need thereof. The symptoms include a compromised skin barrier, inflammation, itchiness sensation, burning sensation, formation of eczematous lesions, and oxidative stress. In some embodiments, the present disclosure provides a kit comprising any of the above-disclosed compositions.

According to another embodiment, the method of the present disclosure further includes the step of orally administering to the subject a dietary supplement containing L-histidine. In some embodiments, the method stimulates filaggrin production in the skin of a subject. In some embodiments, the method stimulates FLG gene synthesis and/or OVOL1 gene synthesis in the skin of a subject. In some embodiments, the skin of the subject suffers from a chronic inflammatory skin disorder. In some embodiments, the skin of the subject suffers from a loss of function mutation in gene FLG. In some embodiments, the present disclosure provides a kit comprising (i) any of the above-disclosed compositions; and (ii) a dietary supplement containing L-histidine.

According to another embodiment, the method of the present disclosure further includes the step of orally administering a dietary supplement containing at least one antioxidant and L-histidine. In some embodiments, the present disclosure provides a kit comprising (i) any of the above-disclosed compositions; and (ii) a dietary supplement comprising at least one antioxidant and L-histidine. Examples of suitable antioxidants include, but are not limited to, Vitamins B, C, D, and E, beta-carotene, selenium, coenzyme Q10, quercetin, glutathione, resveratrol, astaxanthin, lipoic acid, zinc, plant extracts with antioxidant activity, and an aromatic compound. Exemplary aromatic compounds include, but are not limited to, flavonoids, anthocyanins, flavonols, and terpenes.

In some embodiments, the antioxidant comprises zinc. Loss of OVOL1 function, which is potentially impacted by disruptions in zinc availability within the body, has been linked to impaired skin barrier function and an increased susceptibility to chronic inflammatory skin conditions like psoriasis.

Zinc is a crucial micronutrient that acts as a gatekeeper for immune function, essential for developing and activating immune cells like T-cells and natural killer cells. It strengthens defenses by regulating innate and adaptive immunity, reducing inflammation, providing antioxidant protection, and helping to maintain the integrity of skin and mucosal barriers. Zinc is also involved in immune cell development and function, thymus regeneration; inflammation control; and wound healing.

The antioxidant may be employed in amounts ranging from about 0.1 to about 20% by weight, such as from about 1 to about 15% by weight, and from about 2 to about 10% by weight, all weights based on the total weight of the dietary supplement. In some embodiments, the antioxidant is employed in an amount corresponding to its daily recommended value.

According to yet another embodiment, the dietary supplement described herein comprises a mixture of: (a) Buddleja globosa (matico) leaf extract, (b) Aristotelia chilensis (maqui) leaf extract, Ungi molinae (murta) leaf extract, and (d) L-histidine.

Buddleja globosa leaf extract is derived from the leaves of the orange ball buddleja, also known as “matico,” a species of flowering plant endemic to Chile and Argentina. The extract has been found to contain glycosidic flavonoids and phenylethanoids such as verbascoside, iridoids, triterpenoids, and di- and sesquiterpenoids, together with two caffeic acid derivatives. These compounds have shown promise in wound healing due to their ability to promote fibroblast growth, with a strong antioxidant effect. This particular leaf extract is also rich in stigmasterol, an unsaturated plant sterol found in plant oils.

The Buddleja globosa leaf extract is preferably employed in an amount of from about 0.1 to about 5% by weight, or from about 0.5 to about 3% by weight, or from about 1 to about 2% by weight, based on the total weight of the dietary supplement composition. In some embodiments, the Buddleja globosa leaf extract is present in the dietary supplement composition at an amount of about 0.1 to about 5 mg, or about 0.5 to about 3 mg, or about 1 to 2 mg. This extract is commercially available from N-Active EIRL, under the trade name EthniCare® MATICO.

Aristotelia chilensis leaf extract is derived from the leaves of a small dioecious evergreen tree in the Elaeocarpaceae family native to South America in the Valdivian temperate rainforests of Chile, which also goes by the name maqui. The extract has been found to contain high amounts of anthocyanins, indole alkaloids, and flavonoids. These compounds serve as a source of antioxidants that help to neutralize free radicals and protect the skin's DNA.

The Aristotelia chilensis leaf extract is preferably employed in an amount of from about 0.5 to about 20% by weight, or from about 1 to about 10% by weight, or from about 2 to about 5% by weight, based on the total weight of the dietary supplement composition. In some embodiments, the Aristotelia chilensis leaf extract is present in the dietary supplement composition at an amount of about 0.5 to about 20 mg, or about 1 to about 10 mg, or about 2 to about 5 mg. This extract is commercially available from N-Active EIRL under the trade name EthniCare® MAQUI.

Ugni molinae leaf extract is derived from the leaves of a woody evergreen shrub from the myrtaceae family commonly found in Chile and is also known by its Spanish name “murta.” The extract has been found to contain various phenolic compounds including gallic acid, catechin, quercetin, myricetin, and kaempferol. These compounds have been found to possess strong antioxidant activity against ROS production, lipid peroxidation, and superoxide anion production.

The Ugni molinae leaf extract is preferably employed in an amount of from about 0.1 to about 5% by weight, or from about 0.5 to about 3% by weight, or from about 1 to about 2% by weight, based on the total weight of the dietary supplement composition. In some embodiments, the Ugni molinae leaf extract is present in the dietary supplement composition at an amount of about 0.1 to about 5 mg, or from about 0.5 to about 3 mg, or from about 1 to about 2 mg. This extract is commercially available from N-Active EIRL, under the trade name EthniCare® MURTA.

Weight ratios of Aristotelia chilensis leaf extract to Buddleja globosa leaf extract to Ugni molinae leaf extract can range from about 1:1:1 to about 6:1:1, about 1:1:1 to about 5:1:1, about 1:1:1 to about 4:1:1, or about 1:1:1 to about 3:1:1. In some embodiments, the ratio of the Aristotelia chilensis leaf extract to Buddleja globosa leaf extract to Ugni molinae leaf extract is about 1:1:1, about 2:1:1, about 3:1:1, about 4:1:1, about 5:1:1, or about 6:1:1.

All three plant extracts (i.e., Aristotelia chilensis leaf extract, Buddleja globosa leaf extract, and Ugni molinae leaf extract) are derived from plant leaves utilizing a solvent extraction process for recovering the respective leaf extracts. A pre-made blend of all three plant extracts utilized by the invention of the present disclosure is commercially available from N-Active SpA, a Chilean company located in Santiago, Chile under the tradename EthniCare® M3 Powder Plus.

L-histidine is an essential amino acid used by the body in the biosynthesis of proteins. It is a key building block of two key skin barrier components, namely, filaggrin protein that provides structure to the skin barrier and the natural moisturization factor complex that helps attract and retain moisture in skin. L-histidine is commercially available from Fifth Nutrisupply Inc, located in Montclair, California.

The L-histidine may be employed in amounts ranging from about 0.05 to about 10 mg, or from about 0.1 to about 5 mg, or from about 0.2 to about 4 mg, all weights based on the total dry weight of the dietary supplement composition.

According to an embodiment of the present disclosure, the method involves daily application of the topical skincare composition, coupled with daily oral administration of the dietary supplement.

According to yet another embodiment, the method further includes applying a therapeutic amount of a bacteriophage which targets Staphylococcus aureus bacterium, onto the skin. In some embodiments, the bacteriophage topical product further complement the invention of the present disclosure. An example of a suitable bacteriophage for use by the present disclosure includes, but is not limited to, Staphylococcus phage JD419 as described in Feng et al., Front. Microbiol. 12:602902 (2021). The precise amount of bacteriophage used to kill the Staphylococcus aureus bacterium present on a person's skin can be determined by those skilled in the art.

The topical bacteriophage is preferably applied onto the skin prior to applying the composition of the present disclosure.

According to yet another embodiment, the method further includes orally administering a synbiotic supplement containing a therapeutically effective amount of: (a) a probiotic blend of bacterial components containing at least: (i) Lactobacillus helveticus, (ii) Lactobacillus plantarum, (iii) Bifidobacterium longum, and (iii) Streptococcus thermophilus; and (b) at least one plant-based, antioxidant-rich source of fiber. In some embodiments, the use of this type of synbiotic dietary supplement, in combination with the topical composition of the present disclosure, enables symptoms associated with eczema such as gut inflammation and gut microbiome dysbiosis to be ameliorated, while simultaneously enhancing ceramide production in the skin. In some embodiments, the present disclosure provides a kit comprising any of the above-disclosed compositions and the probiotic supplement.

In yet another embodiment, the method of the present disclosure further includes topical application of a probiotic serum containing live Lactobacillus plantarum lactobacilli. In some embodiments, application of the probiotic serum helps to reduce skin inflammation and balance the skin microbiome. In some embodiments, the present disclosure provides a kit comprising any of the above-disclosed compositions and the probiotic serum.

### EXAMPLES

The following examples as set forth herein are intended for illustrative purposes only and are not intended to limit the scope of the disclosure in any way, as many variations thereof are possible without departing from the spirit and scope of the disclosure. In the examples, all concentrations are listed as weight percent, unless otherwise specified.

### Example 1

Calendula officinalis stem cell extract in an amount of 2%, an oil infusion of Symphytum officinale extract in an amount of 1%, and a biotech-derived Haberlea rhodopensis extract in an amount of 1% were evaluated, both individually and in combination, to determine what, if any, gene expression effects they may indicate, per the below-indicated protocol.

Reconstructed Human Epidermis (RHE) tissues were obtained from ZenBio (Research Triangle Park, NC; lot #RHE051820) and were used immediately. Tissues were transferred to 6 well plates and were equilibrated for an hour in 1 ml of pre-warmed medium/well ZenSkin provided by the ZenBio. Samples of the above ingredients were then added non-diluted, in triplicates at 3 mg/cm² with the positive displacement pipette and were spread evenly on top of the RHE tissues. Sterile distilled water was the negative control. Treated tissues were allowed to incubate overnight. The results of the gene expression testing fell into three distinct categories.

At the end of the incubation RNA was extracted and purified with RNeasy Mini Kit cat. #74104 from Qiagen (Germantown, MD), using a QiaCube Connect robotic station (Qiagen). Purified total RNA was assessed at 260 nm and 280 nm with a Thermo Fisher Scientific (Waltham, MA) NanoDrop™ Lite Spectrophotometer.

cDNA was prepared using a High-Capacity RNA-to-cDNA™ Kit (Applied Biosystems, Thermo Fisher) and the expression of the genes of interest was measured by real-time quantitative PCR with a BioRad iCycler iQ Detection System using PCR primers from Realtimeprimers (Elkins Park, PA) and Azura View GreenFast qPCR Blue Mix LR available from Azura Genomics (Raynham, MA). Efficiency ΔΔCt method was used for quantification of results, after the normalization of gene expression to HPRT1 and GAPDH (housekeeping genes).

Genes were considered differentially expressed if the p value, as determined by the two-tailed t-test, was ≤0.10 and the modulation was ≥1.8. However, if a modulation >10 is measured, the p value may be <0.5 for statistical significance.

The synergistic influence exhibited by the mixture of the present disclosure on certain genes associated with anti-oxidative function, as compared to each individual ingredient, is shown in Table 1, below.

TABLE 1

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| HMOX1 | — | — | — | — | — | — | 0.01 | 2.9 |
| SOD1 | — | — | — | — | — | — | 0.01 | −1.2 |
| GPX1 | — | — | — | — | — | — | 0.0 | −1.5 |
| NQO1 | 0.01 | −25.1 | 0.02 | −3.6 | — | — | 0.09 | 1.2 |

In the presence of the mixture, antioxidant genes SOD1 and GPX1 were downregulated and HMOX1/NQ01 were activated to provide a more comprehensive ROS reduction mechanism that not only scavenges ROS but protects skin tissue from damage. See, e.g., Wagener et al., Int. J. Mol. Sci. 14(5):9126-9167 (2013). Moreover, HMOX1 counteracts the cytotoxic effects caused by high concentrations of free heme in the dermatitistic lesion and enables the NRF2/HMOX1 axis to mediate anti-inflammatory activity, whereas NQO1 provides NRF2 a target to help detoxify xenobiotic compounds as well as ROS.

The synergistic influence exhibited by the mixture of the present disclosure on certain genes associated with ceramide production, as compared to each individual ingredient, is shown in Table 2, below.

TABLE 2

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| SMPD1 | — | — | — | — | — | — | 0.01 | 1.7 |

Ceramides play a key role in reducing permeability of the skin barrier. In dermatitis, skin lipids may contain less ceramide and ceramide composition may be altered.

SMPD1 was surprisingly upregulated by the mixture to increase ceramide production. The protein encoded by this gene is a lysosomal acid (sphingomyelinase) that converts sphingomyelin to ceramide. See, e.g., Kitatani et al., Cells 10(9):2386 (2021).

The synergistic influence exhibited by the mixture of the present disclosure on certain genes associated with skin barrier repair/restructuring, as compared to each individual ingredient, is shown in Table 3, below.

TABLE 3

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| CDSN | — | — | — | — | — | — | 0.0 | 3.1 |
| FLG | 0.01 | 13.1 | — | 15.6 | — | — | 0.09 | 41.2 |
| GBA | — | — | 0.03 | −1.8 | — | — | 0.08 | 1.5 |
| LCE3D | — | — | 0.01 | 84.4 | 0.1 | 2.7 | 0.00 | 6.7 |
| LOR | — | — | — | — | — | — | 0.01 | 4.1 |

Skin barrier structure and integrity genes of Table 3, associated with a properly functioning stratum corneum, were significantly upregulated in the presence of the mixture as compared to the individual components, especially FLG which is typically under-expressed in skin suffering from atopic dermatitis. Note that this includes both structural protein genes and binding protein genes.

CDSN codes for corneodesmosin and plays a vital role in the structural and functional integrity of the epidermis and hair follicle integrity by preventing the rupture of corneodesmosome, i.e., the main intercellular adhesive structures in the stratum corneum that are transformed from desmosomes at the most superficial layer of the stratum granulosum of the epidermis. The major compositional distinction as compared to desmosomes is the presence of corneodesmosin in the extracellular portion. CDSN was upregulated by the mixture, thereby enhancing the structural integrity of the stratum corneum through improved adhesion, which is helpful for improving lesions in individuals suffering from AD and psoriasis. See, e.g., Ishida-Yamamoto et al., Cell Tissue Res. 360(3):477-482 (2015) and Gordon et al., Pediatr. Dermatol. 39(2):268-272 (2022).

FLG was significantly upregulated (more than the sum of the individual components, indicating synergistic amplification) which is known to increase the production of pro-filaggrin, a pre-cursor to filaggrin, a critically important protein for skin barrier structure, and under produced in those with atopic dermatitis.

LOR was upregulated to produce the skin barrier protein loricrin, which is a major component of the skin's cornified envelope. Links between loricrin and other components of the envelope, like filaggrin, hold the corneocytes together and help give the stratum corneum its structural strength.

GBA, an enzyme involved in ceramide production, was also significantly upregulated (compared to downregulated in comfrey alone) evidencing the ability of the mixture to enhance the structural integrity of the stratum corneum.

LCE3D is a gene that encodes a protein that contributes to the structural stability of the cornified envelope and acts as an antimicrobial peptide in the skin. Among its related pathways is keratinization as it is involved in the formation of corneocytes and skin barrier maintenance. Note that in psoriasis, LCE3B and LCE3C are suppressed or missing. LCE3D is one of several genes that are significantly correlated with the main infiltration cell of atopic dermatitis (AD). See, e.g., Peng et al., Clin. Cosmet. Investig. Dermatol. 14:437-453 (2021).

The synergistic influence exhibited by the mixture of the present disclosure on certain genes associated with inflammation reduction, as compared to each individual ingredient, is shown in Table 4, below.

TABLE 4

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| EDN1 | 0.16 | 4.3 | 0.07 | 4.7 | — | — | 0.03 | −2.7 |
| ICAM1 | — | — | — | — | — | — | 0.08 | 2.5 |
| IL4 | | | | | | | 0.36 | −49.5 |
| IL8 | — | — | — | — | — | — | 0.15 | 2.1 |
| IRF4 | — | — | — | — | — | — | 0.1 | −1.1 |
| AGER | — | — | — | — | — | — | 0 | −1.8 |
| AHR | — | — | — | — | 0.01 | 1.2 | 0.04 | −1.1 |

Inflammation modulation of atopic dermatitis: AHR/NRF2 also play critical roles in the maintenance of skin homeostasis. Specific disruption of AHR function in the skin has been found to be associated with the pathogenesis of atopic dermatitis. See, e.g., Edamitsu et al., Antioxidants (Basel) 11(2):227 (2022).

AGER was downregulated in the presence of the mixture, evidencing a reduction in the production of cell surface receptors for advanced glycation end-products (RAGE) that can cause an unfavorable proinflammatory state. See, e.g., Serveaux-Dancer et al., Dis. Markers 2019:6067353 (2019) and Budu-Aggrey et al., Nature Comm. 14:6172 (2023).

AHR was downregulated in the presence of the mixture. Without intending to be bound by theory, this downregulation is believed to evidence a reduction in immune mediated skin responses based on AHR's ability to inhibit: (i) autophagy, (ii) extracellular matrix degeneration, and (iii) oxidative stress. See, e.g., Salminen, Cell. Mol. Life Sci. 79:489 (2022).

EDN1 was downregulated in the presence of the mixture, which evidences a reduction in itch response as this gene is associated with the production of endogenous pruritogens [endothelin (ET)-1] in the skin. Elevated plasma ET-1 were significantly correlated with AD clinical severity, itch intensity, and serum IgE levels. See, e.g., Tsybikov et al., Allergy Asthma Proc. 36(4):320-324 (2015) and Nakahara et al., Curr Treat. Options Allergy 6:156-163 (2019).

ICAMI gene was upregulated in the presence of the mixture, evidencing an increase in endothelial cell activation by acting as a leukocyte adhesion molecule thereby helping the affected area fight infection and/or inflammation caused by mechanical/frictional damage such as by scratching. See, e.g., Marinović Kulišić et al., Life 13:933 (2023) and Werfel, J. Invest. Dermatol. 129(8):1878-1891 (2009).

IRF4 gene was downregulated in the presence of the mixture, evidencing a reduction in the inflammatory cascade of NLRP3-mediated IL-33 cytokines such as those triggered in AD lesions, as IRF4 interacts with NLRP3 to control IL-33 expression in epithelial cells. See, e.g., Zheng et al., Cell Disease Death 12:871 (2021).

IL4 gene was significantly downregulated in the presence of the mixture, evidencing a reduced production of the IL-4 cytokine that may impair wound healing by decreasing the production of fibronectin, driving inflammation and barrier disruption by decreasing the expression of genes that contribute to the skin's barrier function and innate immune defense. In addition, IL-4 promotes the differentiation of naïve CD4+ T cells into Th2 cells which, in turn, stimulate the production of several inflammatory cytokines (IL-4, IL-13, IL-5 and IL-19) that are involved in the recruitment of eosinophils, basophils and mast cells and in the release of allergic mediators resulting in itching and burning. See, e.g., Chiricozzi et al., Immunotargets Ther. 9:151-156 (2020).

IL8 gene was upregulated in the presence of the mixture, evidencing production of the IL-8 cytokine that is an attractant for neutrophils and T-cells and stimulates the migration of keratinocytes for wound healing. IL-8 is typically suppressed in patients with AD because it is inhibited by TH2 cytokines. See, e.g., Nomura et al., J. Immunol. 171(6):3262-3269 (2003) and Fania et al., Int. J. Mol. Sci. 23(5):2684 (2022).

The synergistic influence exhibited by the mixture of the present disclosure on certain genes associated with skin barrier homeostasis, as compared to each individual ingredient, is shown in Table 5, below.

TABLE 5

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| PPARD | 0.03 | 7.5 | 0.01 | 3.5 | 0.00 | 19.3 | 0.2 | 4.9 |

PPARD remained upregulated in the presence of the mixture, evidencing an acceleration in wound healing of a structurally impaired (wounded) skin barrier. PPARD has been found to inhibit keratinocyte apoptosis and promote the re-epithelialization of the skin by enhancing keratinocyte adhesion and migration. See, e.g., Blunder et al., Int. J. Mol. Sci. 22 (14): 7354 (2021).)

The synergistic influence exhibited by the mixture of the present disclosure on certain genes associated with cell death (apoptosis), as compared to each individual ingredient, is shown in Table 6, below.

TABLE 6

| | | | | | | | Mixture: | |
|---|---|---|---|---|---|---|---|---|
| | | | | | | | 2% Calendula | |
| | | | | | | | officinalis | |
| | | | | | | | 1% Symphytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| CSF2 | 0.0 | 5.5 | — | — | — | — | 0.36 | 48.2 |
| KITLG | 0.37 | −1.4 | 0.06 | 2.3 | 0.36 | 5.3 | 0.36 | 45.6 |

CSF2 (Colony-stimulating factor 2) was significantly upregulated in the presence of the mixture to produce an endogenous damage signal that promotes the therapeutic effects of mesenchymal stem cells by enhancing their multi-lineage differentiation and migratory capacities. CSF2 also stimulates the proliferation and differentiation of granulocytes and macrophages for addressing infection. As a result, its stimulation is believed to enhance the skin barrier's response to atopic dermatitis flareups. See, e.g., Park et al., Mol Therapy 27(6):1087-1100 (2019).

The synergistic influence exhibited by the mixture of the present disclosure on certain genes associated with tissue remodeling/wound healing, as compared to each individual ingredient, is shown in Table 7, below.

KITLG (stem cell factor (SCF) or mast cell growth factor) encodes the ligand for the KIT receptor tyrosine kinase. It plays a role in epidermal homeostasis and melanocyte development. It regulates skin pigmentation by controlling the proliferation, migration, and survival of melanocytes and influences melanin distribution. In atopic dermatitis, inflammatory cytokines and vasoactive intestinal peptide can cause epidermal keratinocytes to produce stem cell factor.

TABLE 7

| | | | | | | | Mixture: | |
|---|---|---|---|---|---|---|---|---|
| | | | | | | | 2% Calendula | |
| | | | | | | | officinalis | |
| | | | | | | | 1% Sympytum | |
| | | | | | | | officinale | |
| | 2% Calendula | | 1% Symphytum | | 1% Haberlea | | 1% Haberlea | |
| | officinalis | | officinale | | rhodopensis | | rhodopensis | |
| Gene | T-Test | Regulation | T-Test | Regulation | T-Test | Regulation | T-Test | Regulation |
| BMP4 | — | — | — | — | — | — | 0.07 | 2.0 |
| GRHL3 | 0.06 | 1.7 | — | — | — | — | 0.01 | 5.2 |
| PAX3 | — | — | — | — | — | — | 0.07 | 1.8 |
| SERPINH1 | — | — | — | — | — | — | 0.01 | −1.5 |
| TIMP2 | — | — | — | — | — | — | 0 | −1.4 |

Various tissue remodeling and wound healing genes that help mitigate alterations in skin barrier structure in regions suffering from AD or psoriatic inflammation were influenced by the mixture, as compared to each individual ingredient.

BMP4 was upregulated by the mixture. This gene has been observed as an upstream regulator in skin inflammatory disease lesions like AD and psoriasis which activates the RHO family of GTPases, important regulators for diverse cellular processes, including cellular polarization and morphogenesis as well as rearrangement of the actin skeleton in cells to facilitate their removal by phagocytosis. This upregulation enables the clearance of dying keratinocytes for wound healing. See, e.g., Coleman and Olson, Cell Death Differ. 9:493-504 (2002) and Bang et al., Sci Report 12:5889 (2022).

GRHL3-transcription factor, which activates gene expression programs required for cell adhesion, lipid production, cornified envelope formation and protein crosslinking, was significantly upregulated by the mixture (almost three times more than in Calendula alone showing amplificatory synergy). It is essential for skin barrier formation and adult epidermal repair. This upregulation helps to reinforce and restore the stratum corneum. See, e.g., Klein et al., PLoS Genet. e1006745 (2017).

PAX3 was upregulated by the mixture. The protein made by the PAX3 gene directs the activity of other genes that signal neural crest cells to form specialized tissues or cell types including nerve cells and melanocytes. Upregulation of this gene therefore serves to support melanocyte differentiation, survival, proliferation, and migration for healing inflamed regions of skin and local hair follicles. See, e.g., Boudjadi et al., Gene 666:145-157 (2018) and Medic et al., Biochem. Biophys. Res. Comm. 411(4):832-837 (2011).

SERPINH1 which encodes for Hsp47 (heat shock protein), a collagen-specific molecular chaperone that localizes in the endoplasmic reticulum (ER), is indispensable for molecular maturation of collagen by ensuring correct folding of procollagen. This gene was downregulated in the presence of the mixture to decrease procollagen folding in the early stages of wound healing to allow for the fibroblasts to accumulate more components such as hyaluronan and fibronectin in the extracellular matrix and not form scar tissue within the inflammatory lesions. See, e.g., Tyavambiza et al., Bioengineering 9(11):712 (2022) and Ito and Nagata, Seminar Cell Dev. Biol. 62:142-151 (2017).

TIMP2 inhibits several MMPs that cleave a range of substrates that impact structural and cellular aspects of tissue architecture. This gene was downregulated in the presence of the mixture, thereby enabling it to maximize the effectiveness of those MMP genes involved in tissue remodeling/wound healing. See, e.g., Costanzo et al., Pulm. Med. 2022:3632764 (2022).

In summary, the gene expression data of Tables 1-7 establishes the synergy realized by combining the ingredients of the present disclosure when it comes to treating and managing oxidative stress, inflammation, and skin barrier/SC restructuring and repair to help mitigate symptoms associated with inflammatory skin disorders.

### Example 2

A composition in accordance with the present disclosure was prepared having the following ingredients:

| Ingredients | % wt/wt |
|---|---|
| Haberlea rhodopensis | 1.0 |
| Calendula officinalis | 2.0 |
| Symphytum officinale | 2.5 |
| Padina pavonica Thallus | 2.0 |
| Colloidal oatmeal | 1.0 |
| emulsifiers | 2.0 |
| Lactobacillus ferment | 3.0 |
| Coconut fermented and/or mixed with Lactobacillus | 2.0 |
| Potassium sorbate | 0.2 |
| 1,3-propanediol | 4.0 |
| Ceramide | 0.01 |
| Sodium benzoate | 0.2 |
| Hydrolyzed sodium hyaluronate | 0.1 |
| Deionized water | 64.9 |
| additives | q.s. |
| Total | 100 |

The composition of Example 2 was clinically tested on a first cohort of test subjects all of whom exhibited active signs of atopic dermatitis including skin flakiness, irritation, itch, and intense dryness, to determine its efficacy in treating and managing the symptoms associated with atopic dermatitis. Each of the test subjects had a SCORAD, a clinical tool used to score atopic dermatitis, measured by a dermatologist of between 25-50. An EASI score which is a tool used to measure the extent (area) and severity of atopic eczema was also calculated by a dermatologist for each test subject. Thirty-four individuals (10 male and 24 female), aged 22-52, were asked to apply the composition twice a day at home, once in the morning and once in the evening, under normal use conditions, as a replacement for their normal skin treatment routine. Skin health and appearance data was collected on both the treated and non-treated areas of each test subject.

SCORAD data involving measurement of atopic exacerbations was obtained by a dermatologist on day 0 (D0) and day 56 (D56). Scoring parameters included the extent, intensity, and subjective evaluation of atopic exacerbations experienced by each test subject. The data showed that 100% of the subjects tested experienced a significant decrease of, on average, 94% in their SCORAD score on D56, a completely unexpected and surprising discovery.

In addition, 97% of subjects tested experienced a significant decrease of 93%, on average, in the extent of atopic exacerbations, 100% of subjects tested experienced, on average, a 94% decrease in the intensity of atopic exacerbations, and 94% of subjects tested experienced, on average, a 96% decrease in the severity of their symptoms based on their subjective evaluation. Moreover, 100% of subjects tested experienced, on average, an 89% decrease in the number of atopic exacerbations present on their body at D56.

The efficacy of the composition of Example 2 was also assessed using the Eczema Area Severity Index (EASI) scoring system for both the treated versus non-treated area. An EASI score is calculated by a dermatologist who assigns a value of from 0-72 (1.1 to 7 indicates mild disease and 7.1 to 21 indicates moderate disease), for each of the following symptoms: erythema, infiltration/papulation, excoriation, and lichenification. The EASI score corresponds to the sum of these values wherein the lower the score, the more efficacious the composition.

Regarding the untreated zone, only 6% of test subjects experienced a statistically insignificant decrease (improvement) in their EASI score at day 28 (D28) and D56.

Conversely, 85% of the test subjects experienced a 52% decrease of their EASI score at D28, and 100% experienced a 95% decrease in their EASI score at D56.

Next, the composition of Example 2 was evaluated to determine its effect on skin moisturization/hydration. Moisturization/hydration data was obtained using a corneometer, wherein an increase in corneometer value corresponds to an increase in moisturization/hydration. Regarding the untreated zone without active lesion, 3% of test subjects experienced a statistically insignificant 1% decrease in cutaneous hydration at D28, and 9% experienced a statistically insignificant 1% decrease at D56.

Conversely, for the treated zone, the data showed that 97% of test subjects experienced a statistically significant increase in cutaneous hydration of more than 42% on day 28; and 100% of test subjects experienced an increase of more than 78% on day 56.

The efficacy of the composition was also evaluated to determine its effect on transepidermal water loss (TEWL), which is indicative of skin barrier health and functionality. The measurements were performed using a Tewameter™ 300® on both the treated and untreated zones on D0, D28, and D56. Regarding the untreated zone without active lesion, 6% of test subjects experienced a statistically insignificant 0% decrease in TEWL at D28; and 9% experienced a statistically insignificant 1% increase in TEWL at D56.

Conversely, for the treated zone, the data showed that 88% of the test subjects experienced a statistically significant 22% decrease in TEWL at D28; and 100% of test subjects experienced a statistically significant 40% decrease in TEWL at D56.

Next, the composition of Example 2 was clinically evaluated to determine its effect on eczematous lesions by measuring its exfoliating effect using a Skin Image Analyser (S.I.A.®) in combination with QuantiSquam® software. The studied parameters included the desquamation index and surface occupied by squamae (in mm2). A decrease in one of these parameters is characteristic of an exfoliating effect.

Regarding the surface occupied by squamae for the non-treated zone, 55% of test subjects experienced a 44% decrease in squamae area at D56.

Conversely, for the treated zone, the data showed that 88% of test subjects experienced a 49% decrease in squamae area at D28, and 87% of those tested experienced a 64% decrease at D56.

The efficacy of the composition was also evaluated with respect to desquamation index which represents the ratio between occupied surface and the thickness of their cellular layers.

For the untreated area, 84% of subjects experienced an average decrease of 45% at D56.

Conversely, 91% of subjects experienced a 51% decrease on D28, and 87% of subjects experienced a decrease of 65% at D56.

A questionnaire, created by the clinical trial center, was given to each test subject for them to subjectively evaluate the soothing efficacy of the composition with respect to the itching and burning sensation they were experiencing.

With respect to itching sensation, 88% of test subjects experienced a 71% decrease in itching sensation at D28, and 91% experienced a decrease of 93% at D56.

Regarding burning sensation, 44% of test subjects experienced an average decrease of 69% in burning sensation at D28, and 53% experienced a decrease of 79% at D56.

### Example 3

The composition of Example 2 was clinically tested on a second cohort of test subjects suffering from atopic dermatitis. In this Example, topical application of the composition was tested in combination with administration of a dietary supplement containing the following ingredients:

| Ingredient | Weight | |
|---|---|---|
| L-Histidine | 4 | grams |
| Aristotelia chilensis leaf extract | 2 | g |
| Ugni molinae leaf extract | 2 | g |
| Buddleja globosa leaf extract | 2 | g |

Each of the test subjects had an initial SCORAD value measured by a dermatologist of between 25-50. An EASI score was also calculated by a dermatologist for each test subject. Thirty-three individuals aged 19-73, were asked to orally consume the dietary supplement once a day, and to apply the composition onto the surface of their skin twice a day at home, once in the morning and once in the evening, under normal use conditions, as a replacement for their normal skin treatment routine. The subjects were also asked to cleanse their skin with an unscented/neutral soap cleanser prior to applying the composition. Skin health and appearance data was collected on both the treated area (composition plus supplement) and non-treated area (supplement only) for each test subject.

SCORAD data involving measurement of atopic exacerbations was obtained by a dermatologist on D0 and D56. Scoring parameters included the extent, intensity, and subjective evaluation of atopic exacerbations experienced by each test subject.

The data corresponding to the treated area showed that 100% of the subjects tested experienced a significant decrease of, on average, 93% in their SCORAD at D56.

In addition, 100% of subjects experienced an average decrease of 94% for extent of atopic exacerbations, 100% of subjects tested experienced an average decrease of 92% for intensity of atopic exacerbations, and 97% of subjects tested experienced an average decrease of 94% in severity of their symptoms based on their subjective evaluation, all at D56. Moreover, 97% of subjects tested experienced, on average, a 92% decrease in the number of atopic exacerbations present on their body at D56.

The efficacy of the combination of the composition of Example 2 and the dietary supplement was also assessed using the Eczema Area Severity Index (EASI) scoring system for both the treated versus non-treated area. An EASI score is calculated by a dermatologist who assigns a value of from 0-72 (with 1.1 to 7 representing mild disease and 7.1 to 21 representing moderate disease), for each of the following symptoms: erythema, infiltration/papulation, excoriation, and lichenification. The EASI score corresponds to the sum of these values wherein the lower the score, the more efficacious the composition.

Regarding the untreated (supplement only) zone, 9% of test subjects experienced a statistically significant 29% decrease (improvement) in their EASI score at 28, and 12% of test subjects experienced a statistically significant 67% decrease (improvement) in their EASI score at D56.

Regarding the treated (composition+supplement) area, 91% of the test subjects experienced a 62% decrease of their EASI score at 28, and 100% of the test subjects experienced a 90% decrease of their EASI score at D56, as compared to D0.

Next, the combination of the composition of Example 2 and the dietary supplement was evaluated to determine its effect on skin moisturization/hydration. Moisturization/hydration data was obtained using a corneometer, wherein an increase in corneometer value corresponds to an increase in moisturization/hydration (i.e., cutaneous hydration).

Regarding the untreated (supplement only) zone, 73% of test subjects experienced a statistically significant 14% increase in cutaneous hydration at D56.

For the treated (composition+supplement) zone, the data showed that 94% of test subjects experienced a statistically significant average increase in cutaneous hydration of 85% at D28, and 91% experienced a statistically significant average increase in cutaneous hydration of 117% at D56.

The efficacy of the combination of the composition of Example 2 and the dietary supplement was also evaluated to determine its effect on transepidermal water loss (TEWL), which is indicative of skin barrier health and functionality. The measurements were performed using a Tewameter™ 300® on both the treated and untreated zones on D0 and D56.

Regarding the untreated (supplement alone) zone, no statistically significant decrease in TEWL was observed.

For the treated (composition+supplement) zone, the data showed that 77% of the test subjects experienced a statistically significant average decrease of 25% in TEWL at D28, and 79% of the test subjects experienced a statistically significant average decrease of 29% in TEWL at D56.

Next, the combination of the composition of Example 2 and the dietary supplement was clinically evaluated to determine its effect on eczematous lesions by measuring its exfoliating effect using a Skin Image Analyser (S.I.A.®) in combination with QuantiSquam® software. The studied parameters included the desquamation index and surface occupied by squamae (in mm2). A decrease in one of these parameters is characteristic of an exfoliating effect.

Regarding the surface area occupied by squamae for the non-treated (supplement only) zone, 72% of test subjects experienced a 56% decrease in squamae surface area at D56.

For the treated (composition+supplement) zone, the data showed that 81% of test subjects experienced a 65% decrease in squamae surface area at D28, and 97% of test subjects experienced an 82% decrease in squamae surface area at D56.

A questionnaire, created by the clinical trial center, was given to each test subject for them to subjectively evaluate the soothing efficacy of the composition plus supplement with respect to the itching and burning sensation they were experiencing.

With respect to itching sensation, 88% of test subjects experienced a 72% decrease in itching sensation at D28, and 97% of test subjects experienced an 88% decrease in itching sensation at D56.

Regarding burning sensation, 30% of test subjects experienced a 56% decrease in burning sensation at D28, and 45% of test subjects experienced an 84% decrease in burning sensation at D56.

Based on the above data, it is undeniably both surprising and unexpected that the compositions of the present invention, both alone and in combination with the dietary supplement, are compellingly efficacious in not only repairing/reinforcing the skin barrier (SC), but also in reducing inflammation, itchiness, and eczematous lesions. The above-referenced examples establish the effectiveness of the disclosed embodiments to treat and manage symptoms associated with inflammatory skin disorders such as dermatitis and, by extension, any other type of chronic inflammatory disorder such as psoriasis. Specifically, the synergistic influence exerted on certain genes associated with (i) skin barrier repair/restructuring, (ii) inflammation reduction/management, and (iii) reduction/management of oxidative stress caused by ROS in the presence, coupled with the completely astonishing clinical data for the compositions on their own and in combination with the dietary supplement, evidence the compelling efficacy of the present invention when it comes to the treatment and management of symptoms associated with such inflammatory skin disorders. Moreover, consistent use of the disclosed embodiments can help reduce reliance on corticosteroids for symptom relief.

What is claimed is:

1. A method of stimulating filaggrin production in skin of a subject suffering from a chronic inflammatory skin disorder and/or suffering from a loss of function mutation in gene FLG, the method comprising: (i) applying a topical composition onto the skin of the subject, the composition comprising: (1) a synergistic blend of at least: (a) Calendula officinalis extract; (b) Symphytum officinale extract; (c) Haberlea rhodopensis extract; and (d) Padina pavonica thallus extract; and (2) a skin protectant active ingredient, and (ii) orally administering to the subject a dietary supplement comprising L-histidine, wherein the composition is capable of increasing ceramide production and reducing permeability of the skin barrier.

2. The method of claim 1, wherein (a) is present in an amount of from about 0.1 to about 5.0% by weight; (b) is present in an amount of from about 0.1 to about 5.0% by weight; (c) is present in an amount of from about 0.1 to about 5.0% by weight; (d) is present in an amount of from about 0.1 to about 5% by weight; and (2) is present in an amount of from about 0.01 to about 5% by weight, all weights based on total weight of the composition.

3. The method of claim 1, wherein (a) is present in an amount of from about 0.5 to about 4% by weight; (b) is present in an amount of from about 0.1 to about 4% by weight; (c) is present in an amount of from about 0.5 to about 4% by weight; (d) is present in an amount of from about 0.5 to about 4% by weight; and (2) is present in an amount of from about 0.02 to about 3% by weight, all weights based on total weight of the composition.

4. The method of claim 1, wherein (a) is present in an amount of from about 1 to about 3% by weight; (b) is present in an amount of from about 0.4 to about 3% by weight; (c) is present in an amount of from about 0.75 to about 2% by weight; (d) is present in an amount of from about 1 to about 3% by weight; and (2) is present in an amount of from about 0.5 to about 2% by weight, all weights based on total weight of the composition.

5. The method of claim 1, wherein the skin protectant active ingredient is selected from colloidal oatmeal, allantoin, and mixtures thereof.

6. The method of claim 1, wherein the composition further comprises at least one humectant.

7. The method of claim 6, wherein the at least one humectant is present in an amount of from about 0.1 to about 10% by weight, based on total weight of the composition.

8. The method of claim 6, wherein the at least one humectant comprises hyaluronic acid.

9. The method of claim 1, wherein the composition further comprises at least one ceramide.

10. The method of claim 9, wherein the at least one ceramide is present in an amount of from about 0.01 to about 0.5% by weight, based on total weight of the composition.

11. The method of claim 9, wherein the at least one ceramide is selected from ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate.

12. The method of claim 1, wherein the L-histidine is employed in an amount of from about 0.05 to about 10 g in the dietary supplement.

13. The method of claim 1, wherein the L-histidine is employed in an amount of from about 0.1 to about 5 g in the dietary supplement.

14. The method of claim 1, wherein the L-histidine is employed in an amount of from about 0.2 to about 4 g in the dietary supplement.

15. The method of claim 1, wherein the dietary supplement composition further includes at least one antioxidant selected from Vitamin C, Vitamin B, Vitamin D, Vitamin E, beta-carotene, selenium, coenzyme Q10, quercetin, glutathione, resveratrol, astaxanthin, lipoic acid, zinc, plant extracts with antioxidant activity, an aromatic compound, and mixtures thereof, optionally wherein the aromatic compound is selected from a flavonoid, an anthocyanin, a flavonol, a terpene, and mixtures thereof.

16. The method of claim 15, wherein the antioxidant is zinc.

17. The method of claim 15, wherein the antioxidant is employed in an amount of from about 0.1 to about 20% by weight, based on total dry weight of the dietary supplement.

18. The method of claim 15, wherein the antioxidant is employed in an amount corresponding to its daily recommended value.

19. The method of claim 15, wherein the antioxidant is employed in an amount of from about 1 to about 15% by weight, based on total dry weight of the composition.

20. The method of claim 15, wherein the antioxidant is employed in an amount of from about 2 to about 10% by weight, based on total weight of the dietary supplement.

21. The method of claim 15, wherein the plant extract having antioxidant activity is a mixture of: (a) Buddleja globosa leaf extract, (b) Aristotelia chilensis leaf extract, and (c) Ungi molinae leaf extract.

22. The method of claim 1, wherein the topical composition and dietary supplement are employed daily.
